(19) United States
(12) Patent Application Publication
(10) Pub. No.: US 2026/0290577 A1
(43) Pub. Date: Sep. 24, 2026

(54) System and Method for Integrated Health Data Processing and Incentivized Health Optimization Across Multi-Tenant Wellness Platforms

(71) Applicant: Moutakilou Yacoubou, The Bronx, NY (US)

(72) Inventor: Moutakilou Yacoubou, The Bronx, NY (US)

(73) Assignee: StrengthLinx LLC, The Bronx, NY (US)

(21) Appl. No.: 19/567,258

(22) Filed: Mar. 16, 2026

Related U.S. Application Data
(60) Provisional application No. 63/774,558, filed on Mar. 19, 2025.

(51) Int. Cl. G16H 40/67; G16H 10/60

(52) U.S. Cl. CPC G16H 40/67; G16H 10/60

(57) ABSTRACT

A computer-implemented system, method, and non-transitory computer-readable medium are disclosed for integrated health data processing and incentivized health optimization across multi-tenant wellness platforms. The system receives health-related data from mobile devices, wearable devices, fitness facilities, nutrition systems, biometric sources, wellness behavior sources, and third-party platforms. A data processing module aggregates and validates the data, a fraud detection module identifies anomalies or unverifiable activity, and a health optimization scoring engine generates individualized scores based on activity, biometric, nutrition, sleep, and wellness metrics. A rewards ledger or incentive engine dynamically allocates rewards, wellness credits, subsidy decisions, or insurance premium discounts based on verified participation and configurable tenant rules. The platform supports insurers, employers, government health programs, fitness partners, and users through secure multi-tenant infrastructure, dashboards, encrypted data exchange, and audit records to improve preventive health engagement and reduce healthcare costs.

### STATEMENT REGARDING FEDERALLY SPONSORED RESEARCH OR DEVELOPMENT

No invention described in this application was made with federal government support, and the United States Government has no rights in the invention.

### TECHNICAL FIELD

The present disclosure relates to computer-implemented healthcare compliance, health data integration, preventive health engagement, behavioral verification, and incentive administration infrastructure. More particularly, the disclosure relates to systems, methods, and non-transitory computer-readable media for multi-modal verification of preventive healthcare participation, health optimization scoring, fraud detection, rewards administration, data security, and multi-tenant platform governance for employers, health insurers, government health programs, fitness facilities, wearable device ecosystems, nutrition systems, and users.

### BACKGROUND OF THE INVENTION

Preventive healthcare programs administered by employers, insurers, government health programs, and wellness providers may encourage users to participate in physical activity, nutrition programs, chronic disease management, preventive screenings, and related wellness activities. Existing platforms often rely on self-reporting, delayed reimbursement, fragmented data sources, limited fraud detection, and manual administration. Such limitations reduce confidence in reported activity, create administrative burden, and prevent insurers, employers, and public health programs from reliably connecting verified preventive behavior to incentives or program benefits. Accordingly, there is a need for an integrated platform that can ingest data from multiple health and wellness sources, verify participation, compute health optimization metrics, detect anomalies or fraud, administer rewards or subsidy decisions, and provide secure multi-tenant access to different stakeholders.

### SUMMARY OF THE INVENTION

In one embodiment, the disclosed system provides a multi-tenant health data processing and incentivized health optimization platform. The platform includes a mobile application, wearable device interfaces, an API gateway, a data processing module, a health optimization scoring engine, a fraud detection module, a rewards ledger, and insurer, employer, government, or administrator portals. The system receives health-related signals including physical activity data, biometric data, nutrition data, sleep data, wellness behavior data, facility attendance data, and other preventive health participation records. The system aggregates and validates such data, computes health optimization scores or compliance metrics, detects fraud or anomalies, and enables incentives including rewards, wellness credits, subsidy determinations, or insurance premium discounts. In some embodiments, data validation includes GPS location verification, QR code check-in, wearable data correlation, biometric pattern analysis, provider data confirmation, document analysis, and anomaly detection. In some embodiments, the system supports chronic disease management, wellness alerts, medication tracking, and secure data exchange.

### BRIEF DESCRIPTION OF THE DRAWINGS

FIG. 1 illustrates an exemplary StrengthLinx system architecture including a user mobile device, mobile application, wearable fitness device, API gateway, data processing module, fraud detection module, health scoring engine, rewards ledger, insurer/admin portal, and data inputs including activity data, biometric data, nutrition data, and wellness metrics.

FIG. 2 illustrates an exemplary health optimization scoring engine that receives physical activity metrics, biometric data, nutrition data, sleep metrics, and wellness behavior metrics, and processes them through data aggregation, weight assignment, and compliance analysis to generate a health optimization score.

FIG. 3 illustrates an exemplary activity fraud detection workflow including receiving activity data, GPS location verification, device correlation validation, biometric pattern analysis, anomaly detection, and classifying activity as valid or flagged.

FIG. 4 illustrates an exemplary nutrition tracking process including manual food logging, barcode scanning, nutrition mobile applications, AI meal recognition, a nutrition database, calorie analysis, macro tracking, and nutrition compliance scoring.

FIG. 5 illustrates an exemplary wearable device data integration process including wearable sensors, a biometric processing module, and platform outputs including heart rate, step count, sleep monitoring, and oxygen level.

FIG. 6 illustrates an exemplary fitness facility verification system including user arrival at a gym, QR code check-in, GPS location verification, facility attendance validation, and verified workout session output.

FIG. 7 illustrates an exemplary rewards ledger system including a rewards database, transaction records, a blockchain or immutable ledger, and a rewards issuance engine.

FIG. 8 illustrates an exemplary multi-tenant wellness platform including insurer, employer wellness program, government health program, and StrengthLinx platform core connections.

FIG. 9 illustrates an exemplary chronic disease management module including diabetes management, hypertension monitoring, obesity intervention, health alerts, and medication tracking.

FIG. 10 illustrates an exemplary mobile application user workflow including activity tracking, nutrition logging, wearable data synchronization, health feedback display, and StrengthLinx platform interaction.

FIG. 11 illustrates an exemplary rewards redemption system including a rewards catalog, points redemption engine, gift cards, fitness equipment, wellness credits, and insurance premium discounts.

FIG. 12 illustrates an exemplary data security framework including user data storage, encryption, secure server infrastructure, secure data transfer, and insurer data access.

FIG. 13 illustrates an exemplary platform data flow architecture including wearable sensors, mobile application, fitness facility data, nutrition applications, a data processing layer, data ingestion, validation and cleaning, analytics, artificial intelligence and machine learning, data storage, user dashboard, insurer portal, rewards system, and admin panel.

FIG. 14 illustrates an exemplary health optimization score calculation architecture including physical activity, biometric data, nutrition data, wellness behavior metrics, a weighted scoring engine, and a health optimization score.

### DETAILED DESCRIPTION OF THE PREFERRED EMBODIMENTS

The following description is provided to enable persons skilled in the art to make and use the disclosed system. The embodiments described herein are illustrative and may be implemented using cloud computing systems, distributed processing environments, mobile devices, wearable devices, fitness facility systems, nutrition platforms, insurer systems, employer wellness systems, government health program systems, and secure data storage environments. The system may be implemented using one or more processors, memory, databases, network interfaces, APIs, event processing services, machine-learning services, and user interfaces.

### I. System Architecture

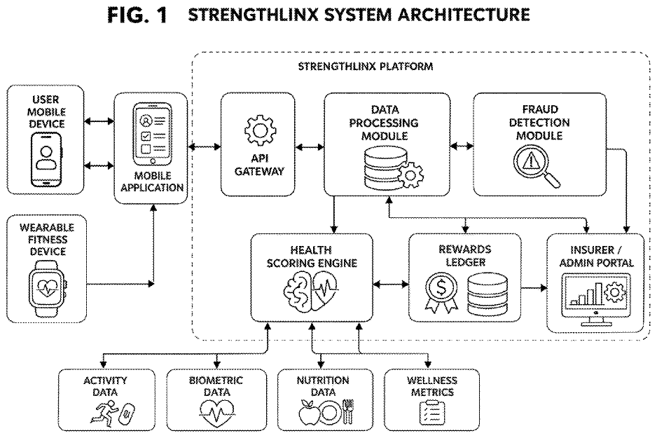

Referring to FIG. 1, a user mobile device and mobile application communicate with the StrengthLinx platform through an API gateway. Wearable fitness devices may transmit biometric and activity data directly or through the mobile application. A data processing module receives and normalizes health-related signals. A fraud detection module evaluates activity integrity. A health scoring engine generates scores based on verified physical activity, biometric data, nutrition data, and wellness metrics. A rewards ledger records incentive transactions, and an insurer/admin portal enables program administrators to configure rules, review analytics, and manage participant benefits.

### II. Health Optimization Scoring

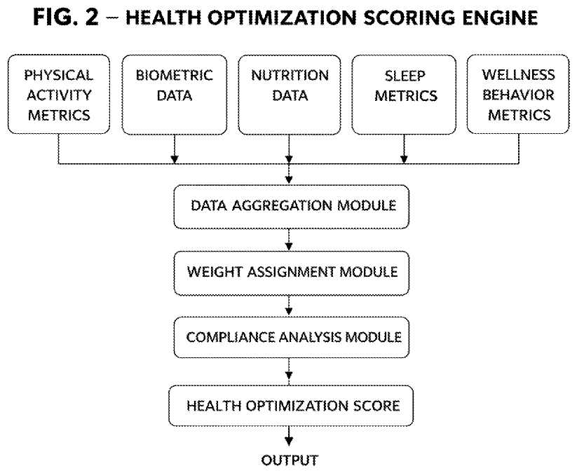

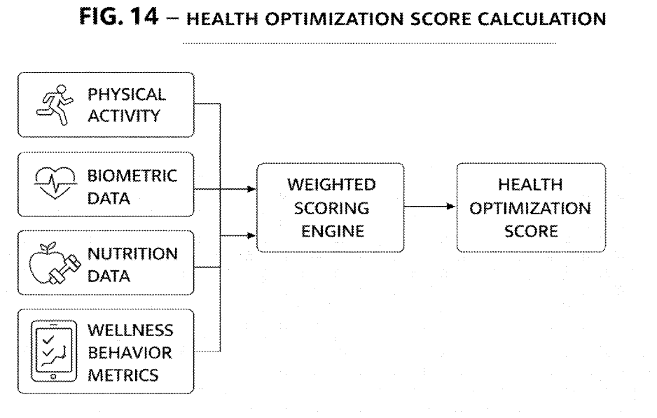

Referring to FIG. 2 and FIG. 14, the health optimization scoring engine may receive physical activity metrics, biometric data, nutrition data, sleep metrics, and wellness behavior metrics. The engine may aggregate data, assign configurable weights to different categories, evaluate compliance with program goals, and output a health optimization score. The score may be used to generate feedback, determine eligibility for rewards, support wellness interventions, or provide reporting to authorized program administrators.

### III. Fraud Detection and Verification

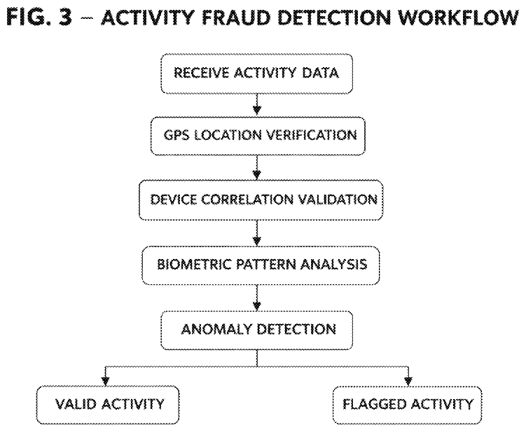

Referring to FIG. 3, the system may detect fraudulent or anomalous activity by receiving activity data, verifying location through GPS or equivalent location signals, validating device correlation, analyzing biometric patterns, and detecting anomalies. Activity may be classified as valid or flagged. Fraud detection may include geolocation comparison, velocity checks, device identity analysis, biometric plausibility checks, frequency analysis, baseline comparison, and administrative review workflows.

### IV. Nutrition Tracking

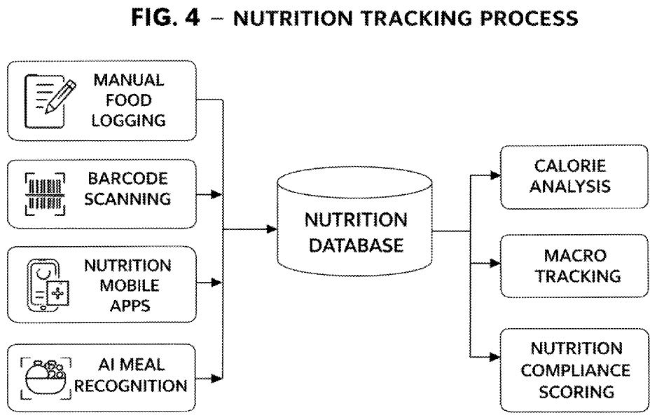

Referring to FIG. 4, nutrition data may be received through manual food logging, barcode scanning, integrated nutrition mobile applications, and AI-based meal recognition. A nutrition database may support calorie analysis, macronutrient tracking, and nutrition compliance scoring. Nutrition compliance may contribute to health optimization scoring, chronic disease management, rewards, or other wellness program outcomes.

### V. Wearable Device Integration

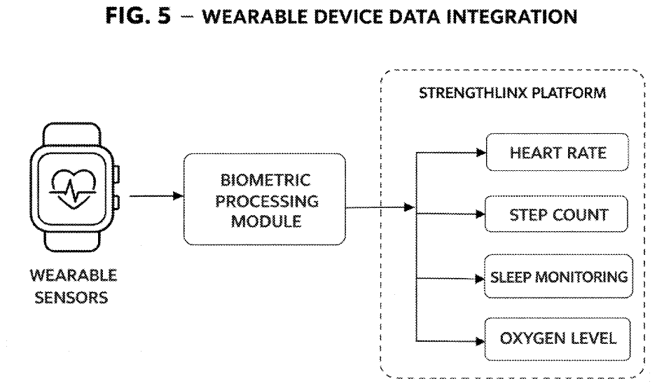

Referring to FIG. 5, wearable sensors may provide biometric and activity signals to a biometric processing module. The platform may process heart rate, step count, sleep monitoring, oxygen level, active minutes, calories, recovery metrics, or other available sensor measurements. Wearable data may be synchronized through device APIs, health platforms, or direct mobile application integrations.

### VI. Fitness Facility Verification

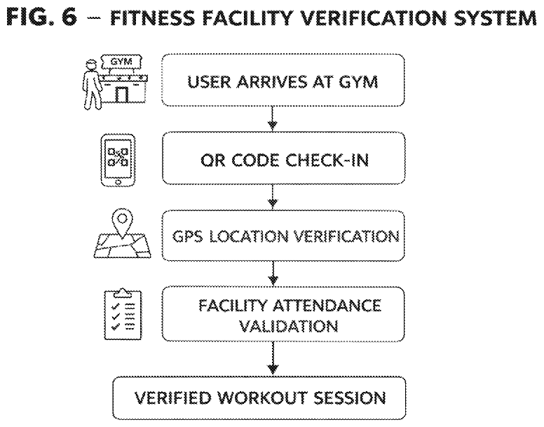

Referring to FIG. 6, a fitness facility verification process may include user arrival at a gym or qualifying facility, QR code check-in, GPS location verification, facility attendance validation, and creation of a verified workout session. Verification may also include NFC, Bluetooth, Wi-Fi proximity, facility management system records, biometric device data, or other corroborating sources.

### VII. Rewards Ledger and Redemption

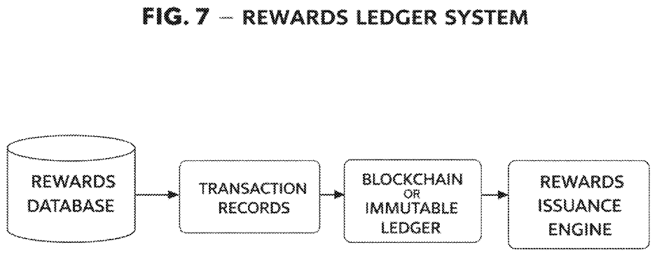

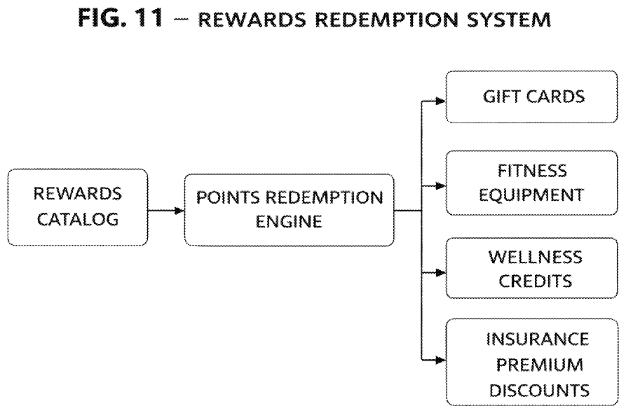

Referring to FIG. 7 and FIG. 11, the rewards ledger may store rewards records, transaction records, and immutable ledger entries. A rewards issuance engine may issue points, wellness credits, gift cards, fitness equipment credits, premium discounts, or other benefits. A points redemption engine may allow users to redeem verified rewards according to program rules.

### VIII. Multi-Tenant Platform

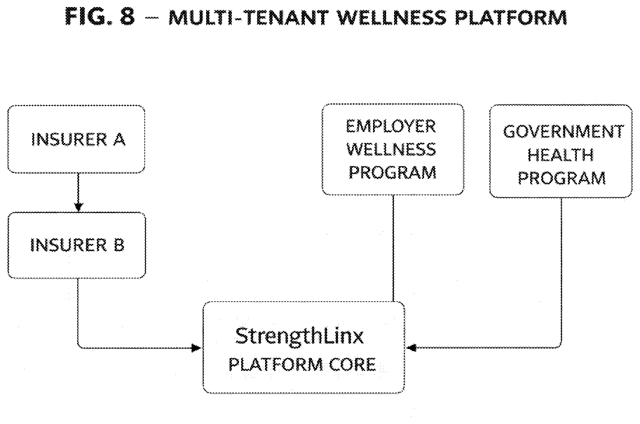

Referring to FIG. 8, the platform may support multiple tenants including insurers, employers, government health programs, fitness partners, and individual users. Each tenant may have configurable program rules, eligibility criteria, reporting views, and data access controls. Tenant isolation may be implemented through account partitioning, role-based access control, encryption, row-level security, segregated databases, or other security mechanisms.

### IX. Chronic Disease Management

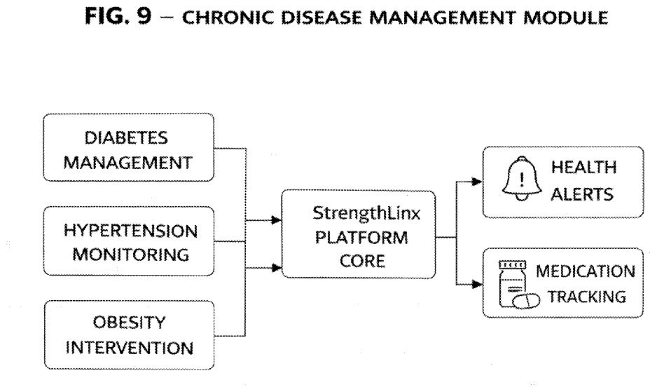

Referring to FIG. 9, the system may support chronic disease management including diabetes management, hypertension monitoring, obesity intervention, health alerts, and medication tracking. The system may process relevant signals to support preventive engagement, adherence monitoring, risk identification, and care coordination.

### X. Mobile Application Workflow

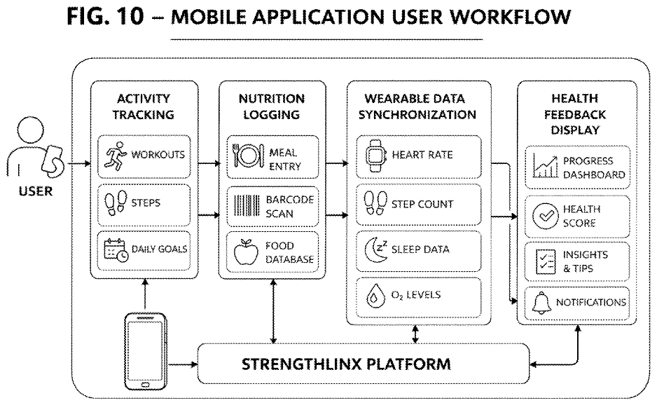

Referring to FIG. 10, a user may interact with the mobile application to track activity, log nutrition, synchronize wearable data, review health feedback, view a progress dashboard, view health scores, receive insights and tips, and receive notifications. The mobile application may communicate with the platform to submit data, receive program status, and display incentive eligibility.

### XI. Data Security

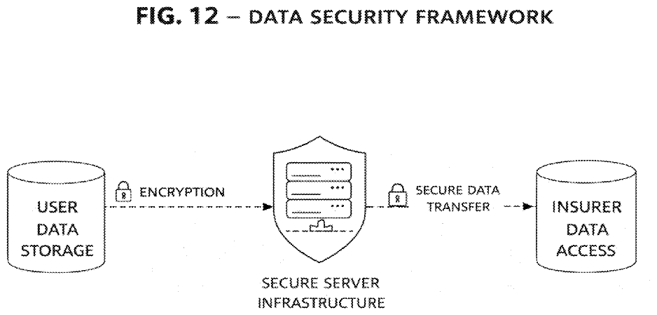

Referring to FIG. 12, the platform may store user data in secure storage, apply encryption, use secure server infrastructure, transfer data securely, and provide authorized insurer or administrator access. Security controls may include encryption in transit and at rest, access logs, consent management, authentication, authorization, and audit trail generation.

### XII. Platform Data Flow and Analytics

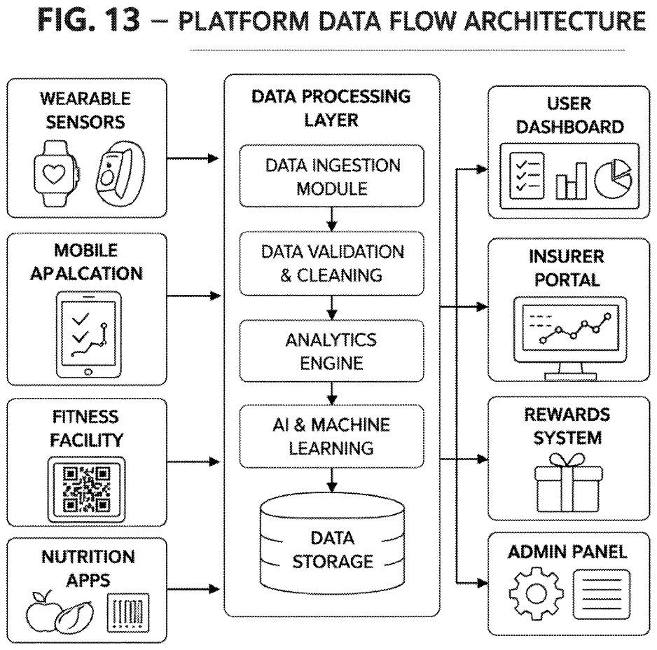

Referring to FIG. 13, wearable sensors, mobile applications, fitness facility systems, and nutrition applications may provide data to a processing layer. The processing layer may include ingestion, validation and cleaning, analytics, artificial intelligence and machine learning, and data storage. Outputs may include user dashboards, insurer portals, rewards systems, and admin panels.

### XIII. Alternative Embodiments

In alternative embodiments, the system may support additional verification channels, including telehealth session metadata, provider API confirmations, document uploads, smart equipment telemetry, camera-based computer vision verification, FHIR API integrations, claims system integrations, HR system integrations, digital wallets, smart-contract escrow systems, and tokenized rewards. The system may be implemented using centralized, distributed, edge, hybrid cloud, or serverless infrastructure.

### XIV. Conclusion

The disclosed system provides technical infrastructure for integrating health data, validating preventive health participation, computing health optimization scores, detecting fraud, securing sensitive data, and administering incentives across multi-tenant wellness ecosystems. The embodiments described herein are not limiting, and variations may be made without departing from the scope of the disclosed invention.

1. A computer-implemented system for integrated health data processing and incentivized health optimization across a multi-tenant wellness platform, the system comprising: one or more processors and memory storing instructions that, when executed by the one or more processors, cause the system to provide a mobile application interface, an API gateway, a data processing module, a fraud detection module, a health optimization scoring engine, a rewards ledger, and an administrator portal; receive health-related data from a plurality of sources including a user mobile device, a wearable fitness device, activity data sources, biometric data sources, nutrition data sources, wellness metric sources, fitness facility systems, and third-party health or wellness platforms; aggregate and validate the health-related data; compute a health optimization score based on weighted analysis of at least physical activity, biometric information, nutrition information, sleep information, and wellness behavior information; determine incentive eligibility according to configurable program rules for at least one tenant organization selected from an insurer, employer wellness program, government health program, or fitness partner; record incentive transactions in the rewards ledger; and provide authorized dashboard access to user, insurer, employer, government program, or administrator interfaces.

2. A computer-implemented method for preventive healthcare incentive governance in a multi-tenant platform serving a plurality of tenant organizations, the method comprising: (a) receiving, by one or more processors, a participation event signal from a participant device, the participation event signal originating from at least one of a GPS location channel, a wearable biometric device channel, a third-party provider API channel, a document upload channel, a telehealth session channel, a nutrition tracking channel, or a fitness facility verification channel; (b) retrieving at least one corroboration signal from a data source independent of the channel that generated the participation event signal; (c) computing a verification trust score by applying a weighted correlation algorithm that evaluates temporal consistency, geographic consistency, physiological plausibility, nutrition consistency, wellness behavior consistency, and provider record authenticity between the participation event signal and the corroboration signal; (d) updating a per-participant eligibility or program status record based on the verification trust score and applicable program eligibility rules; (e) generating a reward, subsidy, premium discount, wellness credit, or other incentive instruction when the verification trust score meets or exceeds a program-configured threshold; and (f) recording the instruction and associated verification data in an audit log.

3. A non-transitory computer-readable medium storing instructions that, when executed by one or more processors, perform operations for health data validation and fraud detection in a preventive healthcare incentive platform, the operations comprising: maintaining a per-participant behavioral baseline model comprising historical distributions of participation timing, geographic location, biometric intensity, nutrition behavior, wellness behavior, and inter-event frequency; receiving a participation event signal; computing temporal anomaly, geographic anomaly, physiological anomaly, nutrition anomaly, wellness behavior anomaly, and frequency anomaly scores by comparing the participation event signal against corresponding distributions in the per-participant behavioral baseline model; computing a composite fraud risk score by applying configurable weights to the anomaly scores; and upon determining that the composite fraud risk score exceeds a configurable threshold, flagging the participation event, restricting or withholding related incentive issuance, and routing the event for automated or manual review.

4. The system of claim 1, wherein the fraud detection module is configured to apply a GPS spoofing detection algorithm to GPS location signals by evaluating signal jitter patterns, location transition velocity, device identity, and consistency with a qualifying facility geofence.

5. The system of claim 1, wherein the data processing module is configured to process participant-submitted document images through an optical character recognition pipeline that extracts provider name, service date, amount, activity description, or program completion information and validates extracted fields against a registered provider directory or program rule set.

6. The system of claim 1, wherein the rewards ledger is configured to route incentive instructions to at least one of a gift card system, wellness credit system, fitness equipment credit system, insurance premium discount system, employer benefit system, health savings account contribution system, or flexible spending account contribution system.

7. The system of claim 1, further comprising a multi-tenant infrastructure layer configured to maintain isolated data environments for a plurality of tenant categories comprising employer tenants, health insurer tenants, government agency tenants, fitness facility tenants, wellness partner tenants, and individual user accounts.

8. The system of claim 7, wherein the multi-tenant infrastructure layer is further configured to enforce data access restrictions using role-based access control, encryption, row-level database security policies, tenant-specific configuration rules, or segregated data stores.

9. The system of claim 1, wherein the health optimization scoring engine is configured to require completion of a minimum number of verified participation events within a configurable enrollment or program activation window as a pre-condition for incentive eligibility.

10. The method of claim 2, wherein computing the verification trust score comprises assigning configurable weights to temporal consistency, geographic consistency, physiological plausibility, nutrition compliance, wellness behavior compliance, and provider record authenticity, and computing the trust score as a weighted sum on a normalized scale.

11. The method of claim 2, further comprising generating, by a compliance or reporting engine, a program compliance score or report by applying applicable policy rules to verified participation records, wherein the program compliance score or report reflects conformance with insurer rules, employer wellness rules, government program rules, privacy requirements, consent requirements, or preventive health program criteria.

12. The non-transitory computer-readable medium of claim 3, wherein maintaining the per-participant behavioral baseline model comprises initializing the model during a calibration period and applying a moving average or machine-learning model update to weight recent verified participation records more heavily than older records in the baseline model computation.
